(19) United States
(12) Patent Application Publication
(10) Pub. No.: US 2025/0384974 A1
(43) Pub. Date: Dec. 18, 2025

(54) Systems and Methods for Optimizing Composite Scores

(71) Applicant: Unlearn.AI, Inc., San Francisco, CA (US)

(72) Inventors: Jonathan Ryan Walsh, El Cerrito, CA (US); Charles Kenneth Fisher, Truckee, CA (US); Arman Sabbaghi, Martinez, CA (US); Daniele Bertolini, Berkeley, CA (US)

(73) Assignee: Unlearn.AI, Inc., San Francisco, CA (US)

(21) Appl. No.: 19/242,683

(22) Filed: Jun. 18, 2025

Related U.S. Application Data
(60) Provisional application No. 63/661,481, filed on Jun. 18, 2024.

(51) Int. Cl. G16H 10/20

(52) U.S. Cl. CPC G16H 10/20

(57) ABSTRACT

Systems and methods for deriving composite scores are illustrated. One embodiment includes a method for optimizing a clinical trial configuration. The method derives item scores for each of a plurality of subjects where each: is based subject data corresponding to a randomized control trial; and answers items from a medical evaluation. The method identifies a parameter to optimize vectors of item weights, wherein: the vectors of item weights are derived using a mean-variance analysis; and each includes a non-negative number. The method determines, for each of the plurality of subjects, an initial composite score, from: one of the at least one vector of item weights; and the plurality of item scores. A resulting collection of composite scores includes the initial composite score determined for each of the plurality of subjects. The method applies the resulting collection of composite scores to implementing a clinical trial.

### CROSS-REFERENCE TO RELATED APPLICATIONS

The current application claims the benefit of and priority under 35 U.S.C. § 119(e) to U.S. Provisional Patent Application No. 63/661,481, entitled “Optimizing Composite Scores for Better Decision Making,” filed Jun. 18, 2024. The disclosure of U.S. Provisional Patent Application No. 63/661,481 is hereby incorporated by reference in its entirety for all purposes.

### FIELD OF THE INVENTION

The present invention generally relates to assessments of potential trial outcomes and, more specifically, the application of historical data to generative models used in assessing potential trial outcomes.

### BACKGROUND

Randomized Controlled Trials (RCTs) are commonly used to assess the safety and efficacy of new treatments, including drugs and medical devices. In RCTs, subjects with particular characteristics are randomly assigned to one or more experimental groups receiving new treatments or to a control group receiving a comparative treatment (e.g., a placebo), and the outcomes from these groups are compared in order to assess the safety and efficacy of the new treatments.

Composite assessments, tests that evaluate disease severity along one or more dimensions through multiple questions or items, are common tools in clinical research. In diseases where patients experience a variety of symptoms affecting one or more bodily systems, composite assessments are an effective way to quantitatively evaluate disease severity. The items on assessments are typically carefully designed by clinical experts and assessments are validated for desirable qualities such as reliability and reproducibility. There are many reasons why someone may use a composite assessment. For example, a clinician may want a reliable, interpretable measure of disease severity, or one that is sensitive to a given patient's particular disease progression. A medical director may want a sensitive measure of disease progression to detect treatment effects, or a way to quickly detect if a drug is effective along any one of many dimensions. A regulator may want a method of assessing disease severity that can be used to benchmark drug efficacy and communicate value to the public.

### SUMMARY OF THE INVENTION

Systems and methods for deriving composite scores are illustrated. One embodiment includes a method for optimizing a clinical trial configuration. The method derives a plurality of item scores for each of a plurality of subjects, wherein the plurality of item scores for a given subject of the plurality of subjects: is based, at least in part, on a first set of subject data corresponding to a randomized control trial; and answers a set of items from at least one medical evaluation. The method identifies, from the plurality of item scores, at least one parameter, wherein the at least one parameter includes an expected value for each of the set of items across the plurality of subjects. The method optimizes, from the at least one parameter, at least one vector of item weights, wherein: the at least one vector of item weights is derived using a mean-variance analysis; and each item weight of the at least one vector of item weights includes a non-negative number. The method determines, for each of the plurality of subjects, an initial composite score. The initial composite score is determined from: one of the at least one vector of item weights; and the plurality of item scores. The mean-variance analysis optimizes the at least one vector of item weights by deriving the item weights that minimize a variance for a resulting collection of composite scores. The resulting collection of composite scores includes the initial composite score determined for each of the plurality of subjects. The method applies the resulting collection of composite scores as a second set of subject data used in implementing a clinical trial. Applying the resulting collection of composite scores includes: determining, based on the resulting collection of composite scores, at least one decision rule for the clinical trial; and deriving, based on the at least one decision rule, one or more of: a desired type-I error rate for the clinical trial; or a desired type-II error rate for the clinical trial.

In a further embodiment, determining, for each of the plurality of subjects, the initial composite score is performed non-linearly.

In another embodiment, the at least one parameter further includes a set of one or more covariance measurements corresponding to the at least one vector of item weights.

In a further embodiment, the set of one or more covariance measurements includes a covariance matrix corresponding to the expected value for each of the set of items across the plurality of subjects.

In another embodiment, each vector of the at least one vector of item weights is uniquely generated for the given subject of the plurality of subjects.

In a further embodiment, the mean-variance analysis is performed at least in part based on a pre-determined target mean composite score; and summing every item weight of the at least one vector of item weights produces a singular numerical constant.

In a further embodiment, the mean-variance analysis includes performing one or more of: minimizing a variance across initial composite scores predicted for the plurality of subjects; or maximizing a ratio of a target mean to a standard deviation across initial composite scores predicted for the plurality of subjects.

In another embodiment, the plurality of item scores is derived based on digital subject data generated by a set of one or more generative models; and at least one of the set of one or more generative models is a neural network trained, at least in part, based on a set of historical data, including one or more of control arm data from historical control arms, patient registries, electronic health records, or real world data.

In a further embodiment, the clinical trial is based, at least in part, on outcome data generated from the set of one or more generative models.

In another embodiment, implementing the clinical trial includes: using the second set of subject data as baseline data for the clinical trial; during at least one future time point in the clinical trial, obtaining subsequent data for the clinical trial. Obtaining the subsequent data for the clinical trial includes determining, for each of the plurality of subjects, an additional composite score. The additional composite score is determined from: the same one, of the at least one vector of item weights, used to determine the initial composite score; and a plurality of subsequent item scores. Obtaining the subsequent data for the clinical trial further includes adding the additional composite score determined for each of the plurality of subjects to the resulting collection of composite scores.

One embodiment includes a non-transitory machine-readable medium including instructions that, when executed, are configured to cause a processor to perform a process for optimizing a clinical trial configuration. The processor derives a plurality of item scores for each of a plurality of subjects, wherein the plurality of item scores for a given subject of the plurality of subjects: is based, at least in part, on a first set of subject data corresponding to a randomized control trial; and answers a set of items from at least one medical evaluation. The processor identifies, from the plurality of item scores, at least one parameter, wherein the at least one parameter includes an expected value for each of the set of items across the plurality of subjects. The processor optimizes, from the at least one parameter, at least one vector of item weights, wherein: the at least one vector of item weights is derived using a mean-variance analysis; and each item weight of the at least one vector of item weights includes a non-negative number. The processor determines, for each of the plurality of subjects, an initial composite score. The initial composite score is determined from: one of the at least one vector of item weights; and the plurality of item scores. The mean-variance analysis optimizes the at least one vector of item weights by deriving the item weights that minimize a variance for a resulting collection of composite scores. The resulting collection of composite scores includes the initial composite score determined for each of the plurality of subjects. The processor applies the resulting collection of composite scores as a second set of subject data used in implementing a clinical trial. Applying the resulting collection of composite scores includes: determining, based on the resulting collection of composite scores, at least one decision rule for the clinical trial; and deriving, based on the at least one decision rule, one or more of: a desired type-I error rate for the clinical trial; or a desired type-II error rate for the clinical trial.

In a further embodiment, determining, for each of the plurality of subjects, the initial composite score is performed non-linearly.

In another embodiment, the at least one parameter further includes a set of one or more covariance measurements corresponding to the at least one vector of item weights.

In a further embodiment, the set of one or more covariance measurements includes a covariance matrix corresponding to the expected value for each of the set of items across the plurality of subjects.

In another embodiment, each vector of the at least one vector of item weights is uniquely generated for the given subject of the plurality of subjects.

In a further embodiment, the mean-variance analysis is performed at least in part based on a pre-determined target mean composite score; and summing every item weight of the at least one vector of item weights produces a singular numerical constant.

In a further embodiment, the mean-variance analysis includes performing one or more of: minimizing a variance across initial composite scores predicted for the plurality of subjects; or maximizing a ratio of a target mean to a standard deviation across initial composite scores predicted for the plurality of subjects.

In another embodiment, the plurality of item scores is derived based on digital subject data generated by a set of one or more generative models; and at least one of the set of one or more generative models is a neural network trained, at least in part, based on a set of historical data, including one or more of control arm data from historical control arms, patient registries, electronic health records, or real world data.

In a further embodiment, the clinical trial is based, at least in part, on outcome data generated from the set of one or more generative models.

In another embodiment, implementing the clinical trial includes: using the second set of subject data as baseline data for the clinical trial; during at least one future time point in the clinical trial, obtaining subsequent data for the clinical trial. Obtaining the subsequent data for the clinical trial includes determining, for each of the plurality of subjects, an additional composite score. The additional composite score is determined from: the same one, of the at least one vector of item weights, used to determine the initial composite score; and a plurality of subsequent item scores. Obtaining the subsequent data for the clinical trial further includes adding the additional composite score determined for each of the plurality of subjects to the resulting collection of composite scores.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates uses for generative models in the analysis of clinical trials in accordance with various embodiments of the invention.

FIG. 2 illustrates using linear models and digital twins used to estimate treatment effects in accordance with several embodiments of the invention.

FIG. 3A illustrates a system for using generative models to estimate treatment effects in accordance with some embodiments of the invention.

FIG. 3B illustrates a system for borrowing information from digital twins to estimate treatment effects in accordance with many embodiments of the invention.

FIG. 4 illustrates a graph depicting the efficient frontier principle, as applied in accordance with numerous embodiments of the invention.

FIG. 5 illustrates a process for optimizing composite scores, performed in accordance with certain embodiments of the invention.

FIG. 6 illustrates a graph depicting the impact of weighted composite scores, performed in accordance with miscellaneous embodiments of the invention, with respect to statistical power.

FIG. 7 illustrates a treatment analysis system that determines treatment effects in accordance with some embodiments of the invention.

FIG. 8 illustrates a treatment analysis element that executes instructions to perform processes that determine treatment effects in accordance with various embodiments of the invention.

FIG. 9 illustrates a treatment analysis application for determining treatment effects in accordance with numerous embodiments of the invention.

### DETAILED DESCRIPTION

Turning now to the drawings, systems and methods implemented in accordance with many embodiments of the invention may be implemented to composite (e.g., risk assessment) scores for use in making evaluations including but not limited to disease severity and/or randomized controlled trial (RCT) results. In this disclosure, randomized controlled trials may also be referred to as experiments and randomized treatments. In obtaining treatment effect inferences from such methods, systems may be configured to incorporate predictions from generative artificial intelligence (AI) algorithms into aspects of RCT analyses. By using generative AI, systems can effectively apply determined (composite) scores to produce estimates including but not limited to diagnostic predictions.

In accordance with some embodiments of the invention, training generative AI algorithms on historical control data (e.g., data obtained from previous RCTs) may enable construction of digital twin generators (DTGs). DTGs may correspond to entities including but not limited to RCT participants. DTGs configured in accordance with certain embodiments of the invention may utilize neural network architectures that can learn conditional generative models of patient trajectories (e.g., based on historical data). For example, DTGs may utilize the baseline covariates of participants (e.g., attributes observed prior to running RCTs) to generate probability distributions for potential control outcomes of the participants (digital twins). Summaries of the probability distributions determined from generative models like DTGs may effectively predict trial outcomes. Doing so can thus be used to improve the quality of output including but not limited to treatment effect inferences, RCT designs, and/or decision rules for treatments.

Data sampled from generative models in accordance with a number embodiments of the invention may be referred to as ‘digital subjects’ throughout this disclosure. In many embodiments, digital subjects can be generated to match given statistics of the treatment groups at the beginning of the study. Digital subjects in accordance with numerous embodiments of the invention can be generated for each subject in a study and the generated digital subjects (“digital twins”) may be used for a counterfactual analysis. In this disclosure, digital twins may refer to digital representations of physical objects, processes, services, and/or environments with the capacity to behave like their counterparts in the real world. In the context of drug and medical studies, digital twins can take the form of representations of the range of potential control (placebo) outcomes of particular clinical trial participants given certain baseline data, such as covariates and characteristics.

In various embodiments, generative models can be used to compute measures of response that are individualized to each patient and that can be used to assess the effects of the treatments. In many embodiments of the invention, estimates for treatment effects may be derived using models including but not limited to prognostic (e.g., machine learning, linear regression) models.

Prognostic models are mathematical models that relate a subject's characteristics now to the risk of a particular future outcome, thereby allowing for RCTs to be efficiently represented. For example, Artificial Intelligence (AI) and Machine Learning (ML) algorithms may enable prognostic models to use historical data to create more efficient trials without introducing bias. When modelling RCTs in a medical context, prognostic models are used to compute prognostic scores, which correlate to the expected outcome or participants with specific pre-treatment covariates if they receive specific control treatments.

In accordance with some embodiments, adjustments may be based on the incorporation of predictor variables including but not limited to prognostic scores determined by models of expected outcomes, and variances of the outcomes. In accordance with many embodiments of the invention, prognostic scores may refer to mean/average values of prognostic models including but not limited to digital twins. Additionally or alternatively, variances of outcomes may refer to variances of prognostic models including but not limited to digital twins which can, in some cases, be implemented as a special classification of AI-based prognostic models.

As suggested above, disease risk assessments may use the personal, genetic and environmental information of participants to determine quantitative value of risk (for developing specific diseases). Systems in accordance with various embodiments of the invention may incorporate scores for establishing estimates (e.g., questionnaires involving a number potential items-“composite assessments”) used to assess different aspects of particular diseases. The individual scores that result from these assessments may be described as item scores. For example, with respect to an Alzheimer's diagnosis, one potential assessment is the Clinical Dementia Rating (CDR) Dementia Staging Instrument, using a 5-point scale to characterize six domains of cognitive and functional performance applicable to Alzheimer disease: Memory, Orientation, Judgment & Problem Solving, Community Affairs, Home & Hobbies, and Personal Care. In such a case, the individual scores for each domain may also be described as the item score. Other common assessments may include but are not limited to Unified Huntington's Disease Rating Scale (UHDRS), Amyotrophic Lateral Sclerosis Functional Rating Scale (ALSFRS), and the Movement Disorder Society-Unified Parkinson's Disease Rating Scale (MDS-UPDRS).

With respect to diseases where patients experience a variety of symptoms affecting one or more bodily systems, composite assessments can effectively evaluate disease severity and/or progression. The items included on assessments are typically carefully designed by clinical experts and assessments are validated for desirable qualities such as reliability and reproducibility. The resulting scores may, additionally or alternatively, be incorporated into generative models including but not limited to digital twin generators in order to provide (e.g., baseline, subsequent) data for predictions of trial outcomes.

Systems and methods in accordance with various embodiments of the invention may produce weighted combinations and/or complex functions of item scores in order to derive composite/total scores that can be implemented by, but are not limited to, generative models. Specifically, these composite scores can be used to determine features including but not limited to which trial endpoints to consider in evaluating treatment effects, and moreover, how much to weigh the resulting value(s) determined at those endpoints.

In the setting of clinical trials, an endpoint used to judge the efficacy of a treatment is the change in score value from baseline. For example, a primary endpoint may be a point used to: evaluate a pre-specified set of one or more values; and use that pre-specified set to detect a statistically significant difference between the treatment and control groups of a clinical trial. For instance, one primary endpoint may be a set point in time (e.g., a few weeks after a trial), after which, the survival of a sick patient (having been placed in the treatment arm) is considered to suggest the treatment had a significant positive impact. A secondary endpoint may be selected to demonstrate additional effects after (success of) the primary endpoint (e.g., a point several years later, used to assess whether quality of life improved from the treatment). Finally, a composite endpoint is an endpoint that is a combination of multiple clinical endpoints. Composite endpoints are commonly used in randomized clinical trials evaluating treatments of diseases including but not limited to Alzheimer's disease (AD), Amyotrophic Lateral Sclerosis (ALS), Huntington's disease (HD) and Parkinson's disease (PD), due to the variety of potential areas of effect associated with these illnesses. For this reason, composite endpoints are essential in evaluating the efficacy (and, especially, the statistical power of clinical trials). Depending on the composite assessment, the population, the accuracy of the expected (or predicted) mean and variance, and assumptions on treatment effects, optimizing the total score can yield substantial increases in statistical power. That said, a major limitation of composite endpoints/assessments stems from equal weighting of all components (i.e., a nominal weighting), regardless of their impact on patients (e.g., quality of life).

As such, processes in accordance with numerous embodiments of the invention may be applied to optimize the score computation from composite assessments for use in prospective and retrospective applications (e.g., in digital twin generator operation). Further, systems may optimize total scores based on item scores including but not limited to those obtained from historical data, current subject data, and/or generative (e.g., machine learning) model-derived output (e.g., digital twins). to the optimization. In doing so, optimization can be tailored for the particular decision making process for which the total scores will be used.

### I. BACKGROUND

As mentioned above, systems and methods configured in accordance with various embodiments of the invention may be directed (but are not limited) to determining disease severity and/or the treatment effects of RCTs. RCT data can include panel data collected from subjects of RCTs and/or can be supplemented with generated subject data. Generated subject data in accordance with a number of embodiments of the invention can include (but are not limited to) digital subject data and/or digital twin data obtained from generative models.

### A. Generative Model Configurations

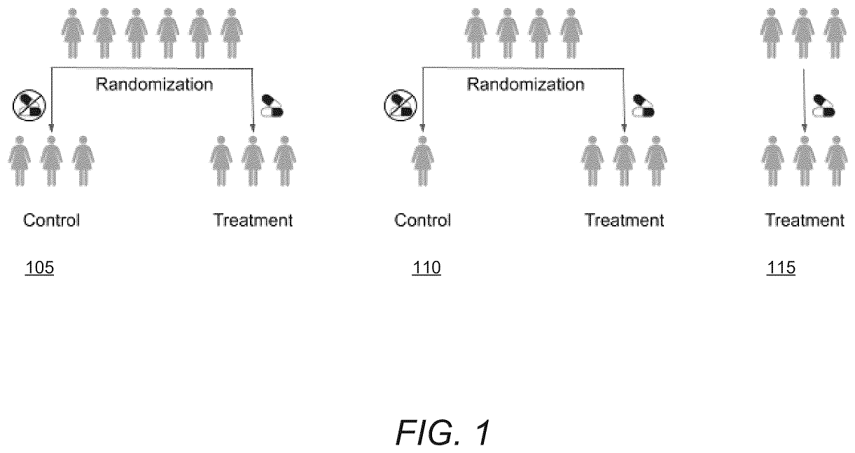

Examples of uses for generative models in the analysis of clinical trials in accordance with various embodiments of the invention are illustrated in FIG. 1. The first example 105 illustrates that generative models, digital subjects, and/or digital twins can be used to increase the statistical power of traditional randomized controlled trials. In the second example 110, generated data is used to decrease the number of subjects required to be enrolled in the control group of a randomized controlled trial. The third example 115 shows that generated data can be used in the external comparator arm of a single-arm trial.

In an RCT, a group of subjects with particular characteristics are randomly assigned to one or more experimental groups receiving new treatments and/or to a control group receiving a comparative treatment (e.g., a placebo), and the outcomes from these groups can be compared in order to assess the safety and efficacy of the new treatments. Without loss of generality, an RCT can be assumed to include i=1, . . . , N human subjects. These subjects are often randomly assigned to a control group and/or to a treatment group such that the probability of being assigned to the treatment group is the same for each subject regardless of any unobserved characteristics. The assignment of subject i to a group is represented by a treatment indicator variable wᵢ. For example, in a study with two groups wᵢ=0 if subject i is assigned to the control group and wᵢ=1 if subject i is assigned to the treatment group. The number of subjects assigned to the treatment group is N_{T}=Σᵢwᵢ and the number of subjects assigned to the control group is N_{c}=N−N_{T}.

In various embodiments, each subject i in an RCT can be described by a vector xᵢ(t) of covariate variables xᵢⱼ(t) at time t. In this description, the notation Xᵢ={xᵢ(t)}ₜ₌₁^{T} denotes the panel of data from subject i and x_{0,i} to denote the vector of data taken at time zero. An RCT is often concerned with estimating how a treatment affects an outcome yᵢ=ƒ(Xᵢ). The function ƒ(·) describes the combination of variables being used to assess the outcome of the treatment. Variables in accordance with a number of embodiments of the invention can include (but is not limited to) simple endpoints based on the value of a single variable at the end of the study, composite scores constructed from the characteristics of a patient at the end of the study, and/or time-dependent outcomes such as rates of range and/or survival times, among others. Approaches in accordance with various embodiments of the invention described herein can be applied to analyze the effect of treatments on one or more outcomes (such as (but not limited to) those related to the efficacy and safety of the treatment).

Each subject has two potential outcomes. When the subject is assigned to the control group wᵢ=0, then yᵢ(0) would be the observed potential outcome. By contrast, when the subject is assigned to receive treatment wᵢ=1, then yᵢ(1) would be the observed potential outcome. In practice, the subject may be assigned to one of the treatment arms such that the observed outcome is Yᵢ=yᵢ(0)(1−wᵢ)+wᵢyᵢ(1). Potential outcomes in accordance with many embodiments of the invention can include various measurements, such as, but not limited to conditional average treatment effect:

$$τ ( {x}_{0} ) = E [ w = 1 , {x}_{0} ] - E [ w = 0 , {x}_{0} ] \qquad ( i )$$

and/or the average treatment effect:

$$τ = E [ τ ( {x}_{0} ) ] = E [ y \vert w = 1 ] - E [ y \vert w = 0 ] . \qquad ( ii )$$

Processes in accordance with several embodiments of the invention can estimate these quantities with high accuracy and precision and/or can determine decision rules for declaring treatments to be effective that have low error rates.

It can be expensive, time-consuming and, in some cases, unethical to recruit human subjects to participate in RCTs. As a result, a number of methods have been developed for using external control arms to reduce the number of subjects required for an RCT. These methods typically fall into two buckets referred to as ‘historical borrowing’ and ‘external control’.

Historical borrowing refers to incorporating data from the control arms of previously completed trials into the analysis of a new trial. Typically, historical borrowing applies Bayesian methods using prior distributions derived from the historical dataset. Such methods can be used to increase the power of a randomized controlled trial, to decrease the size of the control arm, and/or even to replace the control arm with the historical data itself (i.e., an ‘external control arm’). Some examples of external control arms include control arms from previously completed clinical trials (also called historical control arms), patient registries, and data collected from patients undergoing routine care (called real-world data).

The design of RCTs to estimate the effect of new interventions on a given outcome can depend on various constraints, such as (but not limited to) the effect size one wishes to reliably detect, the power to detect that effect size, and/or the desired control of the type-I error rate. Of course, there may also be other considerations such as time and cost, and one may be interested in more than one particular outcome. Although many of the examples described herein are directed at optimizing for a single outcome, one skilled in the art will recognize that similar systems and methods can be used to optimize across multiple outcomes without departing from this invention.

Treatment effect estimators in accordance with many embodiments of the invention may presume a working model for observed outcomes Y=β₀+β₁w+β₂μ+ε where Y, w, and μ are a subject's outcome, treatment status, and prognostic score, respectively and ε is a noise term. In many embodiments of the invention, the noise term for participant i, may be determined such that εᵢ˜N(0, σᵢ²). In accordance with many embodiments of the invention model can be fit via ordinary least-squares and the resulting estimate of β₁, represented by \hat{β}₁, can be taken as the point estimate of the treatment effect. This estimate can be unbiased given treatment randomization without any assumptions about the veracity of the working linear model. Similarly, the assumption-free asymptotic sampling variance \hat{v}²=Var[\hat{β}₁] of this estimate is given by:

$${\hat{v}}^{2} = \frac{{\hat{σ}}_{0}^{2}}{{n}_{0}} + \frac{{\hat{σ}}_{1}^{2}}{{n}_{1}} - \frac{{n}_{0} {n}_{1}}{{n}_{0} + {n}_{1}} {( \frac{{\hat{ρ}}_{0} \hat{{σ}_{0}}}{{n}_{1}} + \frac{{\hat{ρ}}_{1} \hat{{σ}_{1}}}{{n}_{0}} )}^{2} \qquad ( iii )$$

in which

$${\hat{ρ}}_{w} = \frac{Cov [ {Y}_{w} , μ ]}{\sqrt{Var [ μ ] Var [ {Y}_{w} ]}}$$

(where Y_{w} denotes potential outcomes under treatment w=1 and control

$$w = 0 , {\hat{σ}}_{w}^{2} = Var [ {Y}_{w} ] ,$$

n₀ and n₁ the number of enrolled control and treated subjects).

An effect estimate can be declared to be “statistically significant” at level α if a p<α where p=2*(min{Φ(\hat{β}₁/\hat{v}), (1−Φ(\hat{β}₁/\hat{v}))} is the two-sided p-value, \hat{v} is the standard error of \hat{β}₁, and Φ denotes the CDF of the standard normal density. The probability that p<α when, in reality, the treatment effect is β₁ is given by

$$Power = Φ ( {Φ}^{- 1} ( α / 2 ) + \frac{{β}_{1}}{v} ) + Φ ( {Φ}^{- 1} ( α / 2 ) - \frac{{β}_{1}}{v} ) {.}_{{{}^{}}^{}} \qquad ( iv )$$

To power a trial to a given level (e.g., 80%) one must first estimate values for

$${σ}_{w}^{2}$$

and ρ_{w} using prior data (discussed below) or expert opinion. The power formula can then be composed with the variance formula with

$${σ}_{w}^{2}$$

and ρ_{w} fixed at their estimates

$${\hat{σ}}_{w}^{2}$$

and \hat{ρ}_{w}. The resulting function returns power for any values of n₀ and n₁.

The goal of a sample size calculation in the design of a clinical trial (e.g., that uses PROCOVA) can be to estimate n₀ and n₁ required to achieve the required power. However, one needs an additional constraint such as (but not limited to) a chosen randomization ratio n₀/n₁, or minimizing the total trial size n₀+n₁. In this example, the randomization ratio is pre-specified, but the same principles can be easily applied to other situations.

In numerous embodiments, processes for designing a trial can be based on a generative (or prognostic) model. Prognostic models in accordance with many embodiments of the invention can be trained (e.g., based on a prior trial) or pre-trained. Processes can then estimate the variances,

$${σ}_{w}^{2}$$

and correlations, ρ_{w} of the control arm of the trial. One method for obtaining these estimates is to use historical data, such as data from the placebo control arms of previous trials performed on similar populations. In numerous embodiments, estimates can be based on vectors of outcomes for these subjects, gathered during the trials, and their corresponding prognostic, calculated with the prognostic model from each subject's vector of baseline covariates.

In some embodiments, control-arm marginal outcome variance

$${σ}_{0}^{2}$$

can be estimated with the usual estimator

$${\hat{σ}}_{0}^{2} = \frac{1}{{n}^{′} - 1} \sum {( {Y}_{i}^{′} - {\overline{Y}}^{′} )}^{2}$$

where Y̅′ is the sample average. The correlation ρ₀ between μ′ and Y′ can be estimated by \hat{p}₀=Σ(Y′ᵢ−Y̅′)(μ′ᵢ−ū′)/\sqrt{Σ(Y′ᵢ−Y̅′)² Σ(u′ᵢ−μ̅′)²}, the usual sample correlation coefficient. These values may be inflated (for σ₀²) or deflated (for ρ₀) in order to provide more conservative estimates of power.

In certain embodiments, an inflation parameter λ_{w} for the variance and a deflation parameter γ_{w} for the correlation can be applied to sample size calculation. Inflation and deflation parameters can be used to account for the prognostic model. Define the target effect size β₁*, the significance threshold α, the desired power level ζ, fraction of subjects to be randomized to the active arm π, and dropout rate d. Define γ_{w}≥1 and λ_{w}∈[0,1] for w=0,1. Define the variance of the potential outcome under active treatment w in the planned trial as

$${γ}_{w}^{2} {\hat{σ}}_{0}^{2} .$$

so that a large γ_{w} inflates the estimated variance. Similarly, define the correlation between the potential outcome and the prognostic model under active treatment w as λ_{w}\hat{ρ}₀, so that a small λ_{w} deflates the estimated correlation. Then n could be minimized using a numerical optimization algorithm (such as a binary search) such that

$$ζ \geq Φ ( {Φ}^{- 1} ( \frac{α}{2} ) + \frac{{β}_{1}^{*}}{v} ) + Φ ( {Φ}^{- 1} ( \frac{α}{2} ) - \frac{{β}_{1}^{*}}{v} ) , \qquad ( v )$$

with

$${v}^{2} = \frac{1}{n} ( \frac{{γ}_{0}^{2} {\hat{σ}}_{0}^{2}}{1 - π} + \frac{{γ}_{1}^{2} {\hat{σ}}_{0}^{2}}{π} + \frac{{\hat{θ}}^{2} - 2 \hat{θ} * \hat{θ}}{π ( 1 - π )} ) , \hat{θ} = {\hat{ρ}}_{0} {\hat{σ}}_{0} ( ( 1 - π ) {λ}_{0} {γ}_{0} + {πλ}_{1} {γ}_{1} ) ,$$

and \hat{θ}*=\hat{ρ}₀\hat{σ}₀(πλ₀γ₀+(1−π)λ₁γ₁). The minimum sample size can be estimated to be

$${n}_{d} = \frac{n}{1 - d} .$$

Unlike the variances and correlations for a control arm, the corresponding values for the treatment arm can rarely be estimated from data because treatment-arm data for the experimental treatment is likely to be scarce or unavailable. In many embodiments, processes can assume

$${σ}_{0}^{2} = {σ}_{1}^{2}$$

and ρ₀=ρ₁, the latter of which holds exactly if the effect of treatment is constant across the population. It may also be prudent (and conservative) to assume a slightly higher value for

$${σ}_{1}^{2}$$

and a slightly smaller value for ρ₁ relative to their control-arm counterparts.

With the four parameters

$${σ}_{w}^{2}$$

and ρ_{w} specified, the power formula can be computationally optimized over n₀ and n₁ in the desired randomization ratio n₀/n₁ until the minimum values of n₀ and n₁ are found such that the output power meets or exceeds the desired value (e.g., with a numerical optimization scheme).

In many cases, a trial will aim to assess the effect of the intervention on many different outcomes. Processes in accordance with several embodiments of the invention can use multiple prognostic models (e.g., one to predict each outcome of interest) and/or multivariate prognostic models. Depending on the variances of the outcomes, and the accuracy with which they can be predicted, sample size calculations on the various outcomes of interest may suggest different required sample sizes. In this case, one could simply choose the smallest sample size that meets the minimum required statistical power on each of the outcomes of interest.

Systems configured in accordance with some embodiments apply machine learning methods to create simulated subject records. In addition to data from RCTs, generative models in accordance with several embodiments of the invention can link the baseline characteristics x₀ and the control potential outcomes y⁽⁰⁾ through joint probability distributions pθ_{J}(y⁽⁰⁾, x₀) and conditional probability distributions pθ_{c}(x₀), in which θ_{J} and θ_{c} are the parameters of the joint and conditional distributions, respectively. Note that a model of the joint distribution will also provide a model of the conditional distribution, but the converse is not true.

In several embodiments, simulated subject records can be sampled from probabilistic generative models that can be trained on various data, such as (but not limited to) one or more of historical, registry, and/or real-world data. Such models can allow one to extrapolate to new patient populations and study designs.

In some embodiments, generative models may create data in a specialized format—either directly or indirectly—such as the Study Data Tabulation Model (SDTM) and/or the Neyman-Rubin Causal Model to facilitate seamless integration into standard workflows. In a variety of embodiments, generating entire panels of data can be attractive because many of the trial outcomes (such as primary, secondary, and exploratory endpoints as well as safety information) can be analyzed in a parsimonious way using a single generative model.

Systems and methods in accordance with numerous embodiments of the invention can provide various approaches for incorporating data from probabilistic generative models into the analysis of RCTs. In numerous embodiments, such methods can be viewed as borrowing from a model, as opposed to directly borrowing from a historical dataset. As generative models, from which data can be borrowed, may be biased (for example, due to incorrect modelling assumptions), systems and methods in accordance with a number of embodiments of the invention can account for these potential biases in the analysis of RCTs. Generative models in accordance with various embodiments of the invention can provide control over the characteristics of each simulated subject at the beginning of the study. For example, processes in accordance with various embodiments of the invention can create one or more digital twins for each human subject in the study. Processes in accordance with certain embodiments of the invention can incorporate digital twins to increase statistical power and can provide more individualized information than traditional study designs, such as study designs that borrow population-level information and/or that use nearest neighbor matches to patients in historical or real-world databases.

### B. Training Generative Models

In several embodiments, processes can receive historical data that can be used to pre-train generative models and/or to determine prior distribution for (e.g., Bayesian) analyses. Historical data in accordance with numerous embodiments of the invention can include (but is not limited to) control arms from historical data, patient registries, electronic health records, and/or real-world data. In accordance with various embodiments of the invention, the initial training of the models may follow any number of standard machine learning training processes, including but not limited to (e.g., stochastic) maximum likelihood training.

Digital subject data may be generated using generative models. Generative models in accordance with certain embodiments of the invention can be trained to generate potential outcome data based on characteristics of an individual and/or a population. Digital subject data in accordance with several embodiments of the invention can include (but are not limited to) panel data, outcome data, etc. In numerous embodiments, generative models can be trained directly on a specific outcome p(y|x₀). For example, if a goal of using the generative model is to increase the statistical power for the primary analysis of a randomized controlled trial then it may be sufficient (but not necessary) to only use a model of p(y|x₀).

Alternatively, or conjunctively, generative models may be trained to generate panel data that can be used in the analysis of a clinical trial. Data for a subject in a clinical trial is typically in the form of a panel; that is, it describes the observed values of multiple characteristics at multiple discrete timepoints (e.g., visits to the clinical trial site). For example, if a goal of using the generative model is to reduce the number of subjects in the control group of the trial, or as an external comparator for a single-arm trial, then generated panel data in accordance with many embodiments of the invention can be used to perform many or all of the analyses of the trial.

In several embodiments, generative models can include (but are not limited to) traditional statistical models, generative adversarial networks, recurrent neural networks, Gaussian processes, autoencoders, autoregressive models, variational autoencoders, and/or other types of probabilistic generative models. For example, processes in accordance with several embodiments of the invention can use sequential models such as (but not limited to) Conditional Restricted Boltzmann Machines for the full joint distribution of the panel data, p(X), from which any outcome can be computed.

Systems and methods in accordance with numerous embodiments of the invention may determine treatment effects for RCTs using generated digital subject data. Generative models in accordance with many embodiments of the invention can be incorporated into the analysis of an RCT in a variety of different ways for various applications. In many embodiments, generative models can be used to estimate treatment effects by training separate generative models based on data from the control and treatment arms of previous trials. Processes in accordance with many embodiments of the invention can use generative models to generate digital subjects to supplement control arms in RCTs. In certain embodiments, processes can use generative models to generate digital twins for individuals in the control and/or treatment arms. Generative models in accordance with numerous embodiments of the invention may be used to define individualized responses to treatment. Various methods for determining treatment effects in accordance with various embodiments of the invention are described in greater detail herein.

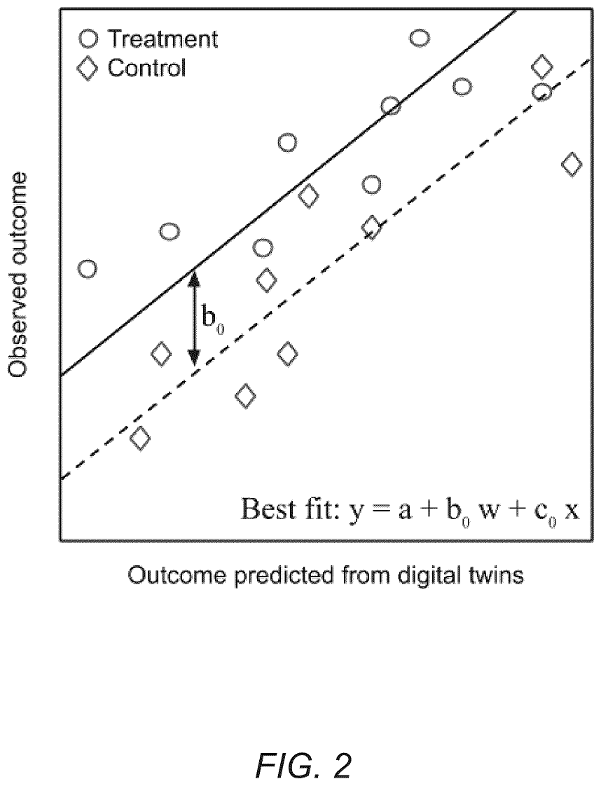

An example of using linear models and digital twins to estimate diagnostic outcomes in accordance with an embodiment of the invention is illustrated in FIG. 2. This drawing illustrates the concept using a simple analysis of a continuous outcome. The x-axis represents the prediction for the outcome from the digital twins, and the y-axis represents the observed outcome of the subjects in the RCT. A linear model is fit to the data from the RCT, adjusting for the outcome predicted from the digital twins. When no interactions are included, then two parallel lines are fit to the data: one to the control group and one to the treatment group. The distance between these lines is an estimate for the treatment effect. Both frequentist and Bayesian methods may be used to analyze the generalized linear model.

In several embodiments, diagnostic predictions and/or treatment effect estimates can be determined by fitting generalized linear models (GLMs) to the generated digital subject data and/or the RCT data. In a number of embodiments, multilevel GLMs can be set up so that the parameters (e.g., the treatment effect) can be estimated through maximum likelihood or Bayesian approaches. In a frequentist approach, one can evaluate the null hypothesis β₀=0, whereas the Bayesian approach may focus on the posterior probability Prob(β₀≥0|data, prior).

In many embodiments, processes can produce estimates by training two new generative models: a treatment model using the data from the treatment group, ρ_{θJ}_{₁}(w₁), and a control model using the data from the control group, ρ_{θJ}_{₀}(w₀). In a variety of embodiments, full panels of data from an RCT can be used to train generative models to create panels of generated data. Such processes can allow for the analysis of many outcomes (including but not limited to primary, secondary, and exploratory efficacy endpoints as well as safety information) by comparing the trained treatment models against trained control models. For simplicity, the notation p(y, x₀) will be used instead of p(X), with the understanding that the former can always be obtained from the latter by generating a panel of data X and then computing a specific outcome y=ƒ(X) from the panel.

In one embodiment, generative models for the control condition (e.g., a Conditional Restricted Boltzmann Machine) can be trained on historical data from previously completed clinical trials. Then, two new generative models for the control and treatment groups can be obtained by solving minimization problems:

$${min}_{{θ}_{{J}_{0}}} \{ - {\sum }_{i} ( 1 - {w}_{i} ) {logp}_{{θ}_{{J}_{0}}} ( {Y}_{i} , {x}_{0 , i} \vert {w}_{0} ) + {λ}_{0} D ( {p}_{{θ}_{{J}_{0}}} , {p}_{{θ}_{J}} ) \} \qquad ( vi )$$

$${min}_{{θ}_{{J}_{1}}} \{ - {\sum }_{i} {w}_{i} {logp}_{{θ}_{{J}_{1}}} ( {Y}_{i} , {x}_{0 , i} \vert {w}_{1} ) + {λ}_{1} D ( {p}_{{θ}_{{J}_{1}}} , {p}_{{θ}_{J}} ) \} \qquad ( vii )$$

in which λ₀ and λ₁ are prior parameters that describe how well pre-trained generative models describe the outcomes in the two arms of the RCT, and D(·,·) is a measure of the difference between two generative models such as (but not limited to) the Kullback-Leibler divergence.

In some embodiments, the new generative models may, additionally or alternatively, be conditional generative models (e.g., Conditional Restricted Boltzmann Machines). The estimate for the result can then be computed as:

$$\hat{τ} = \int dy {dxyp}_{{θ}_{{J}_{1}}} ( y , x \vert {w}_{1} ) - \int dy dx {yp}_{{θ}_{{J}_{0}}} ( y , x \vert {w}_{0} ) . \qquad ( viii )$$

In several embodiments, (e.g., treatment effect) estimates can be computed by drawing samples from the control and treatment models and comparing the distributions of the samples. Processes in accordance with some embodiments of the invention can further tune the computation of estimates by adjusting for the uncertainty in the estimates. In several embodiments, the uncertainty (σ₊) can be obtained using bootstraps by repeatedly resampling the data from the RCTs (with replacement), training the updated generative models, and/or computing the corresponding estimates (wherein the uncertainty is the standard deviation of these estimates). In a number of embodiments, point estimates (e.g., for the treatment effect, for the disease risk) and the estimates for uncertainty can be used to perform hypothesis tests in order to create decision rules.

In numerous embodiments, processes can begin with distributions π(θ_{J}) for the parameters of the generative model (e.g., obtained from a Bayesian analysis of historical data). Then, posterior distributions for θ_{J0} and θ_{J1} can be estimated by applying Bayes rule:

$$\begin{matrix}log π ( {θ}_{{J}_{0}} ) = constant + {\sum }_{i} ( 1 - {w}_{i} ) {p}_{{θ}_{{J}_{0}}} ( {Y}_{i} , {x}_{i} \vert {w}_{0} ) + {λ}_{0} log π ( {θ}_{J} ) \\ log π ( {θ}_{{J}_{1}} ) = constant + {\sum }_{i} {w}_{i} {p}_{{θ}_{{J}_{1}}} ( {Y}_{i} , {x}_{i} \vert {w}_{1} ) + {λ}_{1} log π ( {θ}_{J} ) .\end{matrix} \qquad ( ix )$$

In certain embodiments, point estimates can be calculated as the mean of the posterior distribution

$$\hat{τ} = \int dy dxd {θ}_{{J}_{1}} {yp}_{{θ}_{{J}_{1}}} ( y , x \vert {w}_{1} ) π ( {θ}_{{J}_{1}} ) - \int dy dxd {θ}_{{J}_{0}} {yp}_{{θ}_{{J}_{0}}} ( y , x \vert {w}_{0} ) π ( {θ}_{{J}_{1}} ) , \qquad ( x )$$

where the uncertainty is the variance of the posterior distribution

$${δ}^{2} τ = \int dy dxd {θ}_{{J}_{1}} {y}^{2} {p}_{{θ}_{{J}_{1}}} ( y , x \vert {w}_{1} ) π ( {θ}_{{J}_{1}} ) - \int dy dxd {θ}_{{J}_{0}} {y}^{2} {p}_{{θ}_{{J}_{0}}} ( y , x \vert {w}_{0} ) π ( {θ}_{{J}_{1}} ) - {\hat{τ}}^{2} . \qquad ( xi )$$

As above, point estimates and estimates for their uncertainty can be used to perform a hypothesis test in order to create a decision rule in accordance with certain embodiments of the invention. Processes in accordance with a variety of embodiments of the invention can train conditional generative models ρ_{θJ}_{₁}(x₀, w₁) and ρ_{θJ}_{₀}(x₀, w₀), as opposed to (or in conjunction with) joint generative models, in order to make estimate that are conditioned on the baseline covariates x₀.

It can be difficult to determine the operating characteristics of decision rules based on these methods. Specifically, extensive simulations can be required in order to estimate the type-I error rate (i.e., the probability that an ineffective treatment would be declared to be effective) and/or the type-II error rate (i.e., the probability that an effective treatment would be declared ineffective). Well-characterized operating characteristics are required for many applications of RCTs and, as a result, this approach is often impractical. Generative models that rely on modern machine learning techniques are typically computationally expensive to train. As a result, using the bootstrap and/or Bayesian methods to obtain uncertainties required to formulate reasonable decision rules can be quite challenging.

### C. Applying Generative Models

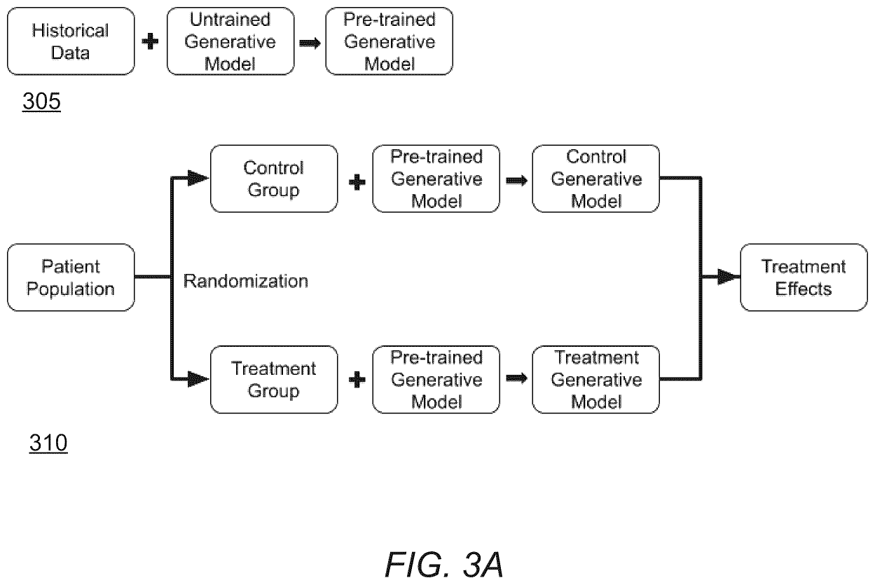

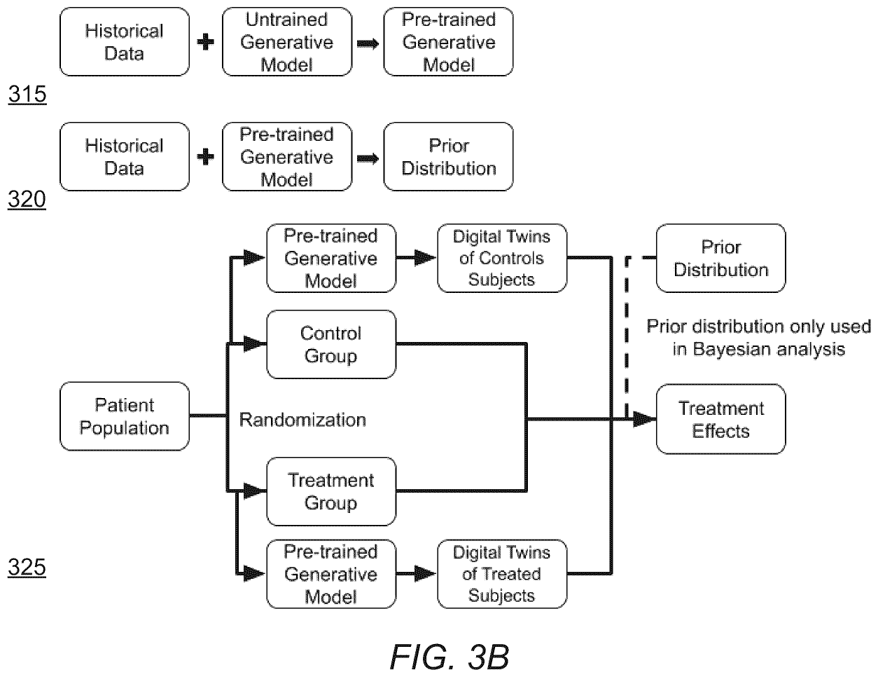

An example of using generative models to estimate treatment effects in accordance with an embodiment of the invention is illustrated in FIG. 3A. In the first stage 305, an untrained generative model of the control condition is trained using historical data, such as (but not limited to), data from previously completed clinical trials, electronic health records, and/or other studies. In the second stage 310, a patient population is randomly divided into a control group and a treatment group as part of a randomized controlled trial. Patients from the population can be randomized into the control and treatment groups with unequal randomization in accordance with a variety of embodiments of the invention. In this example, two new generative models are trained: one for the control group and one for the treatment group. In certain embodiments, control and treatment generative models can be based on a pre-trained generative model but can be additionally trained to reflect new information from the RCT. Outputs from the control and generative models can then be compared to compute the treatment effects. In several embodiments, Bayesian methods and/or bootstrapping may be used to estimate uncertainties and decision rules based on p-values and/or posterior probabilities.

Some methods estimate treatment effects using GLMs while adjusting for covariates. For example, one may perform a regression of the final outcome in the trial against the treatment indicator and a measure of disease severity at the start of the trial. As long as the covariate was measured before the treatment was assigned in a randomized controlled trial, then adjusting for the covariate will not bias the estimate for the treatment effect in a frequentist analysis. When using covariate adjustment, the statistical power is a function of the correlation between the outcome and the covariate being adjusted for; the larger the correlation, the higher the power.

In theory, the covariate that is most correlated with the outcome that one could obtain is an accurate prediction of the outcome. Therefore, another method to incorporate generative models into RCTs in accordance with a variety of embodiments of the invention is to use generative models to predict outcomes and to adjust for the predicted outcomes in a GLM for estimating the treatment effect. Let Eₚ[yᵢ] and Varₚ[yᵢ] denote the expected value and variance of the outcome predicted for subject i by the generative model, respectively. Depending on the type of generative model, these moments may be computable analytically or, more generally, by drawing samples from the generative model p(x_{0,i}) and computing Monte Carlo estimates of the moments in accordance with a number of embodiments of the invention. The number of samples used to compute the Monte Carlo estimates can be a parameter selected by the researcher. As above, processes in accordance with several embodiments of the invention can use generative models that generate panel data so that a single generative model may be used for analyses of many outcomes in a given trial (e.g., primary, secondary, and exploratory endpoints as well as safety information). In a number of embodiments, rather than predictions for given outcomes, predictions of multiple outcomes derived from a generative model may all be included in a GLM for particular outcomes. Samples drawn from the generative models in accordance with several embodiments of the invention can be conditioned on the characteristics of subjects at the start of the trial, also referred to as digital twins of that subject.

In many embodiments, digital twins can be incorporated into an RCT (e.g., in order to estimate the treatment effect) by fitting a GLM of the form:

$$\begin{matrix}g ( E [ {y}_{i} ] ) = a + ( {b}_{0} + {\sum }_{j} {b}_{j} {x}_{0 , ij} ) {w}_{i} + ( {c}_{0} + {\sum }_{j} {c}_{j} {x}_{0 , ij} ) g ( {E}_{p} [ {y}_{i} ] ) + {\sum }_{j} {d}_{j} {x}_{ij} \\ + ( {z}_{0} + {\sum }_{j} {z}_{j} {x}_{0 , ij} ) {w}_{i} g ( {E}_{p} [ {y}_{i} ] )\end{matrix} \qquad ( xii )$$

in which g(·) is a link function. For example, g(μ)=μ corresponds to a linear regression and g(μ)=log(μ/(1−μ)) corresponds to logistic regression. This framework in accordance with numerous embodiments of the invention can also include Cox proportional hazards models used for survival analysis as a special case. In many embodiments, some of these coefficients may be set to zero to create simpler models. The above equation can be generalized to various applications and implementations. The terms involving the b coefficients represent the treatment effect, which may depend on the baseline covariates x₀. The terms involving the c coefficients represent potential bias in the generative model, which may depend on the baseline covariates x₀. The terms involving the d coefficients represent potential baseline differences between the treatment and control groups in the trial. The terms involving the z coefficients reflect that the relationship between the predicted and observed outcomes may be affected by the treatment. The model can be fit using any of a variety of methods for fitting GLMs. One skilled in the art will recognize that it is trivial to include other predictions from the generative model as covariates if desired.

In accordance with many embodiments of the invention, uncertainty values can be applied in a variety of ways. Processes in accordance with many embodiments of the invention can estimate uncertainties analytically and/or using bootstraps by repeatedly resampling the data (with replacement) and re-fitting the model; the uncertainties can be the standard deviations of the coefficients computed by this resampling procedure. In some embodiments, point estimates and estimates for their uncertainty can be used to perform a hypothesis test in order to create a decision rule.

In several embodiments, variances of the outcomes can be modeled through another GLM that adjusts for the variance of the outcome that is predicted by the generative model. For example, variances in accordance with many embodiments of the invention can be modeled as follows:

$$\begin{matrix}G ( Var [ {y}_{i} ] ) = α + ( {β}_{0} + {\sum }_{j} {β}_{j} {x}_{0 , ij} ) {w}_{i} + ( {γ}_{0} + {\sum }_{j} {γ}_{j} {x}_{0 , ij} ) G ( {Var}_{p} [ {y}_{i} ] \\ + {\sum }_{j} {δ}_{j} {x}_{ij} + ( {ζ}_{0} + {\sum }_{j} {ζ}_{j} {x}_{0 , ij} ) {w}_{i} G ( {Var}_{p} [ {y}_{i} ] )\end{matrix} \qquad ( xiii )$$

in which G(·) is a link function that is appropriate for the variance. For example, G(σ²)=log(σ²) can be used for a continuous outcome. In many embodiments, some of these coefficients may be set to zero to create simpler models. One skilled in the art will recognize that other predictions from the generative model can be included as covariates when desired.

Well-trained generative models in accordance with certain embodiments of the invention can have g(E[yᵢ])≈g(Eₚ[yᵢ]) and G(Var[yᵢ])≈G(Varₚ[yᵢ]) by construction. Therefore, prior knowledge about the coefficients in the GLMs can be used to improve the estimation of the treatment effect. However, machine learning models may not generalize perfectly to data outside of the training set. Typically, the generalization performance of a model is measured by holding out some data from the model training phase so that the held-out data can be used to evaluate the performance of the model. For example, suppose that there are one or more control arms from historical clinical trials in addition to the generative model. Then, the c coefficients in accordance with various embodiments of the invention can be estimated by fitting a reduced GLM on the historical control arm data:

$$g ( E [ {y}_{i} ] ) = a + ( {c}_{0} + {\sum }_{j} {c}_{j} {x}_{0 , ij} ) g ( {E}_{p} [ {y}_{i} ] ) , \qquad ( xiv )$$

for the mean or:

$$G ( Var [ {y}_{i} ] ) = α + ( {γ}_{0} + {\sum }_{j} {γ}_{j} {x}_{0 , ij} ) G ( {Var}_{p} [ {y}_{i} ] ) , \qquad ( xv )$$

for the variance. This is particularly useful in a Bayesian framework, in which a distribution π(a,c) or π(α,γ) can be estimated for these coefficients using the historical data, where the data-driven prior distribution can be used in a Bayesian analysis of the RCT. Essentially, this uses the historical data to determine how well the generative model is likely to generalize to new populations, and then applies this information to the analysis of the RCT. In the limit that π(a,c)→δ(a−0)δ(c−1), then digital twins in accordance with a variety of embodiments of the invention can become substitutable for actual control subjects in the RCT. As a result, the better the generative model, the fewer control subjects required in the RCT. In some embodiments, similar approaches could be used to include prior information on any coefficients that are active when wᵢ=0, including the d coefficients.

Examples of workflows for frequentist and Bayesian analyses of clinical trials that incorporate digital twins to estimate treatment effects in accordance with various embodiments of the invention are described below. For a frequentist case for a continuous endpoint, consider a simple example:

$$E [ {y}_{i} ] = a + {b}_{0} {w}_{i} + {c}_{0} {E}_{p} [ {y}_{i} ] \qquad ( xvi )$$

$$while :$$

$$Var [ {y}_{i} ] = {σ}^{2} \qquad ( xvii )$$

assuming no interactions and homoscedastic errors. One skilled in the art will recognize how this can be applied to the more general case captured by Equation xii and Equation xiii. In numerous embodiments, simple analyses can lead to results that are more easily interpreted. This model implies a normal likelihood,

$${y}_{i} \sim N ( a + {b}_{0} {w}_{i} + {c}_{0} {E}_{p} [ {y}_{\dot{l}} ] , {σ}^{2} ) \qquad ( xviii )$$

such that the model can be fit (e.g., by maximum likelihood). There are two situations to consider: (1) the design of the trial has already been determined by some method prior to incorporating the digital twins such that the digital twins can be used to increase the statistical power of the trial, and/or (2) the trial needs to be designed so that it incorporates digital twins to achieve an efficient design with sufficient power. In the case of a continuous endpoint, the statistical power of the trial will depend on the correlation between yᵢ and Eₚ[yᵢ], which can be estimated from historical data, and is a function of the magnitude of the treatment effect. In a variety of embodiments, analytical formulas can be derived in this special case. Alternatively, or conjunctively, computer simulations can be utilized in the general case.

Once the trial is designed, patients are enrolled and followed until their outcome is measured. In some cases, patients may not be able to finish the trial and various methods (such as Last Observation Carried Forward) need to be applied in order to impute outcomes for the patients who have not finished the trial, as in most clinical trials. In a number of embodiments, GLMs can be fit to the data from the trial to obtain point estimates \hat{b}₀ and uncertainties \hat{σ}_{b}_{₀} for the treatment effect. The ratio \hat{b}₀/\hat{σ}_{b}_{₀} follows a Student's t-distribution which can be used to compute a p-value p_{b}_{₀}, and the null-hypothesis that there is no treatment effect can be rejected if p_{b}_{₀}≤ [symbol] in which [symbol] is the desired control of the type-I error rate. This approach is guaranteed to control the type-I error rate, whereas the realized power will be related to the out-of-sample correlation of yᵢ and Eₚ[yᵢ] and the true effect size.

In the Bayesian case for a continuous endpoint with homoscedastic errors, assume a simple analysis,

$$E [ {y}_{i} ] = a + {b}_{0} {w}_{i} + {c}_{0} {E}_{p} [ {y}_{i} ] \qquad ( xix )$$

$$Var [ {y}_{i} ] = {σ}^{2} \qquad ( xx )$$

In certain embodiments, the simple analysis can lead to results that are more easily interpreted. This model implies a normal likelihood:

$${y}_{i} \sim N ( a + {b}_{0} {w}_{i} + {c}_{0} {E}_{p} [ {y}_{i} ] , {σ}^{2} ) \qquad ( xxi )$$

but processes in accordance with various embodiments of the invention can use a Bayesian approach to fit it instead of the method of maximum likelihood. In particular, with historical data representing the condition wᵢ=0 that was not used to train the generative model, processes in accordance with many embodiments of the invention can fit the model:

$$E [ {y}_{i} ] = a + {c}_{0} {E}_{p} [ {y}_{i} ] \qquad ( xxii )$$

to the historical data in order to derive prior distributions for the analysis of the RCT. To do so, pick a prior distribution π₀(a, c₀, σ²) such as (but not limited to) a Normal-Inverse-Gamma prior or another appropriate prior distribution. As there are no data to inform the parameters of the prior before analyzing the historical data, processes in accordance with several embodiments of the invention can use a diffuse or default prior. In numerous embodiments, Bayesian updates to the prior distribution can be computed from the historical data to derive a new distribution π_{H}(a, c₀, σ²), in which the subscript H can be used to denote that this distribution was obtained from historical data. Processes in accordance with numerous embodiments of the invention can then specify prior distributions π₀(b₀) for the treatment effects. This could also be derived from data in accordance with many embodiments of the invention if it is available, or a diffuse or default prior could be used. The full prior distribution is now π_{H}(a, c₀, σ²)π₀(b₀). In various embodiments, such distributions can be used to compute the expected sample size in order to design the trial, as in typical Bayesian trial designs. Once the trial is designed, patients can be enrolled and followed until their outcome is measured. In some cases, patients may not be able to finish the trial and various methods (such as Last Observation Carried Forward) can be applied in order to impute outcomes for the patients who have not finished the trial, as in most clinical trials.

In numerous embodiments, GLMs can be fit to obtain posterior distributions π_{RCT}(a, b₀, c₀, σ²) for the parameters. As in a typical Bayesian analysis, the treatment can be declared effective if Prob(b₀≥0) exceeds a pre-specified threshold in accordance with a number of embodiments of the invention.

There are advantages and disadvantages to the frequentist and Bayesian methods that are captured through these simple examples. The frequentist approach to including digital twins in the analysis of an RCT leads to an increase in statistical power while controlling the type-I error rate. If desired, it is also possible to use the theoretical increase in statistical power to decrease the number of subjects required for the concurrent control arm, although this cannot be reduced to zero concurrent control subjects. The Bayesian approach borrows more information about the generalizability of the model used to create the digital twins (e.g., from an analysis of historical data) and, as a result, can increase the power much more than the frequentist approach. In addition, the use of Bayesian methods in accordance with numerous embodiments of the invention can enable one to decrease the size of the concurrent control arm even further. However, the increase in power/decrease in required sample size can come at the cost of an uncontrolled type-I error rate. Therefore, processes in accordance with many embodiments of the invention can perform computer simulations of the Bayesian analysis to estimate the type-I error rate so that the operating characteristics of the trial can be described.

As a final example, it is helpful to consider a simple case in which a GLM is also used for the variance. For example, consider the models

$$E [ {y}_{i} ] = a + {b}_{0} {w}_{i} + {c}_{0} {E}_{p} [ {y}_{i} ] \qquad ( xxiii )$$

$$and$$

$$log Var [ {y}_{i} ] = α + {β}_{0} {w}_{i} + {γ}_{0} {log}_{p} Var [ {y}_{i} ] , \qquad ( xxiv )$$

$$which reflect the likelihood :$$

$${y}_{i} ∼ N ( a + {b}_{0} {w}_{i} + {c}_{0} {E}_{p} [ {y}_{i} ] , {e}^{α + {β}_{0} {w}_{i} + {γ}_{0} l o g V a {r}_{p} [ {y}_{i} ]} ) . \qquad ( xxv )$$

Models in accordance with a number of embodiments of the invention can allow for heteroskedasticity in which the variance of the outcome is correlated with the variance predicted by the digital twin model, and in which the variance may be affected by the treatment. In several embodiments, a system of GLMs can be fit (e.g., using maximum likelihood, Bayesian approaches, etc.), as was the case for the simpler model. One skilled in the art will clearly recognize that one could also include the interaction or other terms in order to model more complex relationships if necessary. In addition, one skilled in the art will also recognize that including interactions can lead to estimates of conditional averages in addition to standard averages.

An example of borrowing information from digital twins to produce estimates in accordance with an embodiment of the invention is illustrated in FIG. 3B. In the first part 315, a generative model of the control condition is trained using historical data from previously completed clinical trials, electronic health records, or other studies. In the second part 320, if the analysis to be performed is Bayesian, predictions from the generative model are compared to historical data that were not used to train the model in order to obtain a prior distribution capturing how well the predictions generalize to new populations. A frequentist analysis does not need to obtain a prior distribution. In the third part 325, a randomized controlled trial is conducted (potentially with unequal randomization), digital twins are generated for each subject in the trial, and all of the data are incorporated into a statistical analysis (including the prior from part 320 if the analysis is Bayesian) to estimate the treatment effects. Bayesian methods, analytical calculations, or the bootstrap may be used to estimate uncertainties in the treatment effects, and decision rules based on p-values or posterior probabilities may be applied.

Generative models implemented in accordance with many embodiments of the invention are not limited to use within RCTs. Accordingly, it should be appreciated that applications described herein may also be implemented outside the context of clinical trials. Moreover, while specific generative model configurations are described above in FIGS. 1-3B, any of a variety of model configurations can be utilized to produce digital twin-based estimates as appropriate to the requirements of specific applications.

### II. OPTIMIZING COMPOSITE SCORES

Systems and methods in accordance with numerous embodiments of the invention may be applied based on composite assessments applied to a population P of individuals. In many embodiments of the invention, these composite assessments may be applied at various times and/or to various subpopulations, including but not limited to digital subjects. These composite assessment results may be used to make various decision rules and/or optimize type-I/type-II error. For example, when the population corresponds to the population of a randomized controlled trial, systems may apply the composite assessments by using the change(s) from baseline (i.e., via the total score) over the trial duration to make judgments including but not limited to treatment efficacy. As such, processes implemented in accordance with many embodiments of the invention may be applied to derive total scores to achieve the highest quality decisions possible. Optimizing the resulting decision rules may be a byproduct of optimizing the total scores produced. Therefore, given an objective and constraints, systems in accordance with multiple embodiments may be utilized to define one or more optimization algorithms to arrive at a function that computes the (optimized) total score.

In many embodiments of the invention, optimized total scores may be configured for the population such that a defined target mean (total score) minimizes the standard deviation (total score). In various embodiments, the target mean may be based on but is not limited to historical data analyses and/or generative model (e.g., DTG) output. The optimization can (additionally or alternatively to the total scores) be applied to changes in total scores. In many embodiments of the invention, this optimization may be based, in part, on the concept of modern portfolio theory (or mean-variance analysis). There, the problem is determining the combination of a fixed set of assets in a portfolio with relevant quantities are the rate of return of each asset and the covariance matrix between the asset returns (i.e., the risk).

With respect to that theory, the optimized weights may reflect the concept of the efficient frontier, the set of optimal weights across a range of target mean values. In accordance with many embodiments of the invention, the efficient frontier may operate based on a set of properties.

First, for each target mean, there is a unique set of weights that gives a total score that minimizes the variance (in the resulting composite scores determined for the subjects). The set of all such weights over the corresponding target means may describe the efficient frontier.

Second, there is a single set of weights (a portfolio) with minimum variance, which is less than the variance of any individual item (asset).

Third, there is (another) single set of weights that minimizes the ratio of the resulting target mean to the standard deviation (which will be referred to as the “Sharpe” or “MSDR” point in this disclosure).

Fourth, the point on the efficient frontier with the largest variance is simply a weighting where the single item with the largest variance receives the total weight, and all other items have weight 0, with all items on the efficient frontier having a variance between this point and the minimum variance point.

Lastly, any other weight combination not on the efficient frontier is suboptimal to one on the frontier, in that there exists a weight combination on the frontier with the same mean but larger variance. Therefore, as a brief description, the efficient frontier shows the minimum variance that can be achieved for a target mean.

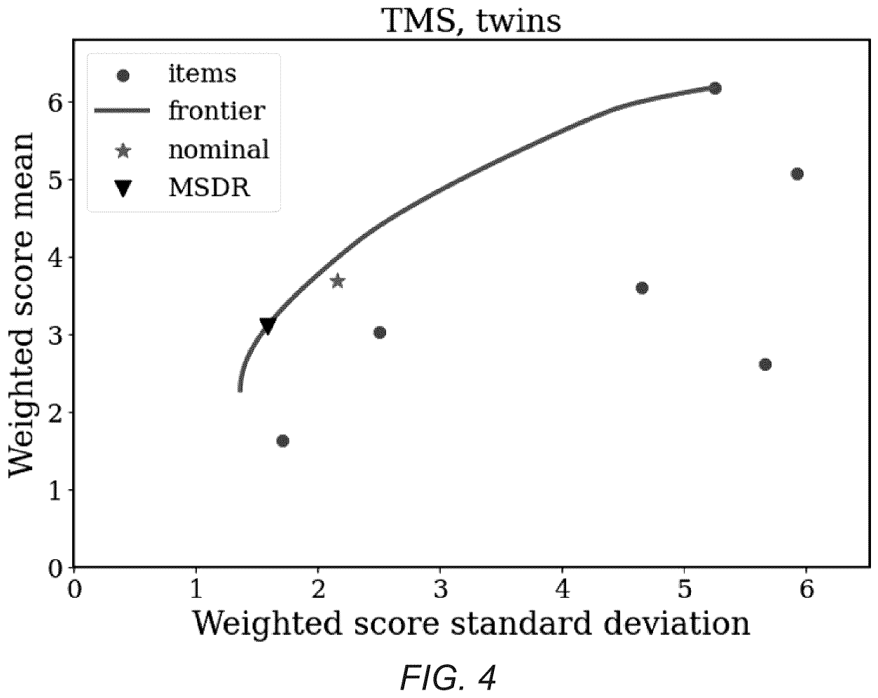

A graph depicting the efficient frontier principle, as applied in accordance with numerous embodiments of the invention, is illustrated in FIG. 4. The example depicted reflects total motor scores (TMS) calculated based on data collected from individuals with Huntington's disease. The TMS is a sub-score determined as part of the Unified Huntington's Disease Rating Scale (UHDRS). Specifically, the score is a total score determined based on a set of “31 items assessing oculomotor, bradykinesia/rigidity, dystonia, chorea, and gait/balance . . . rated from zero to four, with zero indicating normal findings and four indicating severe abnormalities . . . [so t]he range of the Total Motor Score (TMS) is 0 to 124, with higher scores indicating more severe motor impairment,” as is disclosed in Winder, Jessica Y et al. “Assessment Scales for Patients with Advanced Huntington's Disease: Comparison of the UHDRS and UHDRS-FAP.” Movement disorders clinical practice vol. 5,5 527-533. 24 Aug. 2018, doi:10.1002/mdc3.12646, the disclosure of which, specifically the portions depicting the TMS scoring metric is incorporated by reference in its entirety.

The graph of FIG. 4 depicts a nominal TMS score, dispersed item scores, an efficient frontier curve, and the Sharpe point. The nominal TMS score is depicted as a star to represent a TMS score wherein each item is weighed equally. The items themselves are represented with the circular dots scattered throughout, with each dot representing the TMS score if a specific item received the full weight for the calculation. The curve displayed through the middle of the graph represents the efficient frontier, with each point on the frontier representing a trade-off between signal and noise, depending on trial objectives. As such, each of the (effectively infinite) points in the efficient frontier curve corresponds to a specific reweighting approach (with the set collectively representing the maximum signal possible across a noise set). Finally, the Sharpe point, represented as a triangle on the frontier, represents the point where the minimized variance also minimizes the ratio of the resulting target mean to the standard deviation. The unique Sharpe point on the frontier maximizes the signal-to-noise ratio, representing the optimal point for clinical trial design (i.e., maximum power).

In a clinical trial, this approach may be used to determine a total score that is more statistically powerful than the standard (nominal) definition. Depending on assumptions for how treatment effects arise across items of the total score, systems and methods in accordance with numerous embodiments of the invention can select the point on the efficient frontier that provides the best properties for the total score in powering a study. As such, the (convex) optimization problem may be solved, in accordance with numerous embodiments of the invention, in order to reconfigure the total score weights.

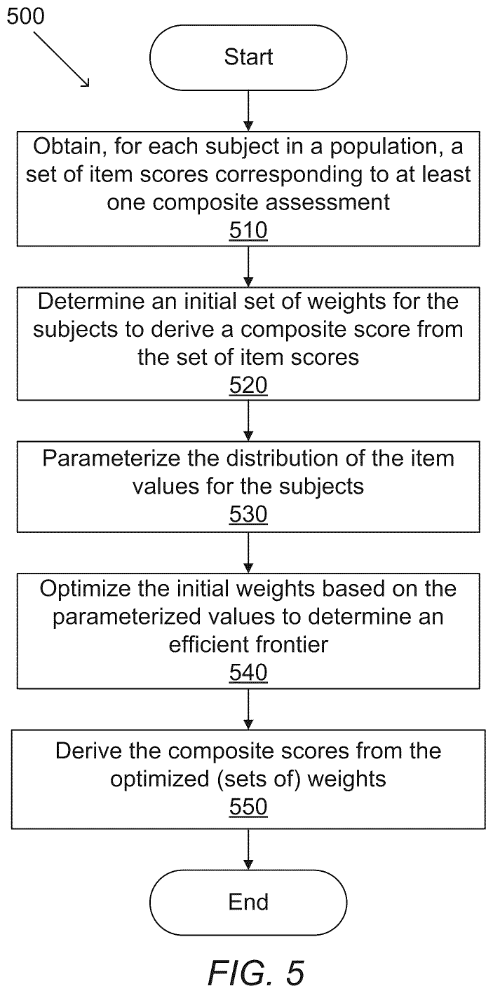

A process for optimizing composite scores, performed in accordance with certain embodiments of the invention, is illustrated in FIG. 5. Process 500 may take the form of an algorithm implemented by generative (e.g., machine learning) models in many cases. In accordance with various embodiments, process 500 may be performed on an individual-level (providing a “personalized composite score” with the weights being independently determined for each potential participant), on a population-level (i.e., with the weights constant across all participants), and/or across subsets of the population (e.g., specifically the control/treatment arm of a trial). Process 500 obtains (510), for each subject in a population, a set of item scores corresponding to at least one composite assessment. In accordance with many embodiments of the invention, the item scores may all be numerical. Additionally or alternatively, the item scores may be normalized in order to adhere to a consistent range, according to the standard of their respective composite assessment(s). Examples of potential composite assessments may include but are not limited to the Clinical Dementia Rating, Pneumonia Severity Index (PSI), and Mini-Mental State Examination (MMSE) assessments. In accordance with a variety of embodiments of the invention, the item scores may be derived based on historical data (i.e., previously gathered composite assessment results). In some such embodiments of the invention, the item scores may be generated from digital twin generators (e.g., based on the above historical data). In miscellaneous cases, the entirety of the population may be represented through actual results, the entirety of the population may be represented through generated results, and/or subsets of the population may be represented through generated results while other subsets are represented through actual results.

Process 500 determines (520) an initial set of weights for the subjects to derive a composite score from the set of item scores. In accordance with multiple embodiments of the invention, a singular set of weights may be used to calculate the composite scores for the entire population. Additionally or alternatively, the weights may be determined (and/or optimized) on a personal basis for each subject. In many cases, each of the items' weights may be set to be non-negative values (wᵢ≥0). This constraint provides interpretability to the total score. It can be assumed, without loss of generality, that each item's score increases with increasing disease severity (reversing any scores that go in the other direction), and hence a positivity constraint on item weights ensures that each item's contribution to the total score increases with increasing (disease) severity.

In various embodiments, the weights may be applied in various linear and/or non-linear manners. The definition of scoring functions may, in accordance with numerous embodiments, be determined through solving objective functions with datasets. For example, certain scoring functions may be defined by neural network(s), including but not limited to using fits to model predictions on reference datasets. In some embodiments of the invention, constraints may be placed on scoring functions including but not limited to monotonicity (when an input xᵢⱼ increases, then f(xᵢ) does not decrease) and calibration (e.g., for a reference population, the average Eᵢ[f(xᵢ)] is a given value) constraints. In accordance with many embodiments of the invention, scoring functions may be used in tandem with personalized (i.e., based upon model predictions for an individual's outcomes) and/or non-personalized (i.e., fixed for all individuals) approaches.

Additionally or alternatively, in many embodiments of the invention, the weights may be applied linearly such that the total score is a weighted sum. In such cases, the total score may follow the formula:

$${s}_{i} = {\sum }_{j = 1}^{n} {w}_{i j} {x}_{ij} ;$$

where, for subject i, xᵢⱼ represents one of n item scores for the subject, wᵢⱼ represents the corresponding (e.g., personalized, constant across subjects) weight, and sᵢ represents the determined total score. When the total score is a weighted sum, its derivation may follow the limitation that the item weights sum to a constant (Σᵢwᵢ=wₜₒₜ). This constraint controls the total weight applied to all items, and sets a normalization common to all choices of item weights.

Process 500 parameterizes (530) the distribution of the item values for the subjects. In numerous embodiments, the derived parameters may include but are not limited to the expected mean and covariance of the items. For prospective applications, systems and methods in accordance with numerous embodiments of the invention may, additionally or alternatively, determine accurate estimates for changes in item scores (rather than item scores themselves). In any event, parameters such as the mean and covariance for the items, can (in a number of embodiments) be provided by generative models. Further, in cases where the reweighting process is performed on an individual level, a mean vector for the items and a covariance matrix for each subject may be utilized (and potentially, the above mean vectors may be combined to obtain a population average). In some embodiments, the covariance of the items may be determined based on the covariance between item means and/or represented as the (i.e., full matrix) covariance between the entire set of items.

Further, as mentioned above, processes in accordance with many embodiments of the invention may be performed for entire populations, subsets of populations, and/or on the individual level. When performed for individuals, the parameters (e.g., the mean and covariance) may be determined over items for singular individuals. As such, systems and methods in accordance with many embodiments of the invention may utilize item-level generative models for single individuals in determining the item scores and/or deriving these parameters.

Process 500 optimizes (540) the initial weights based on the parameterized values to determine an efficient frontier. This optimization may be summarized as minimizing the variance of the total score, across the weights. In many embodiments of the invention, the optimization problem can be framed as minimizing w^{T}Σw, the variance of the total score, across weight vectors w, such that the target mean: w^{T}μ=t, where Σ is the covariance matrix across items, μ is the mean across items, and t is the target mean (total score). In accordance with some embodiments of the invention, the weights may be optimized in order to align with the Sharpe point (i.e., to the single set of weights that minimizes the ratio of the target mean, t, to standard deviation). Additionally or alternatively, the weights may be optimized in order to align with the single set of weights that minimizes the standard deviation. Assumptions under factors including but not limited to treatment effects might be used to determine which (combinations of) optimization bases might be used.

Process 500 derives (550) the composite scores from the optimized (sets of) weights. As suggested above, in miscellaneous instances, optimized weights may be constant across the entirety of the population. In doing so, the optimized weights may be applied to the individual item scores for each individual participant accordingly. Additionally or alternatively, subsets of participants may have constant optimized weights (e.g., the entire control arm of a given trial, a particular demographic). Additionally or alternatively, in various instances, both the item scores and the optimized weights may be personalized, with the combination producing personalized composite scores. Naturally, this is an optimal case, where economically feasible, and can effectively increase the statistical power of the trial even further.

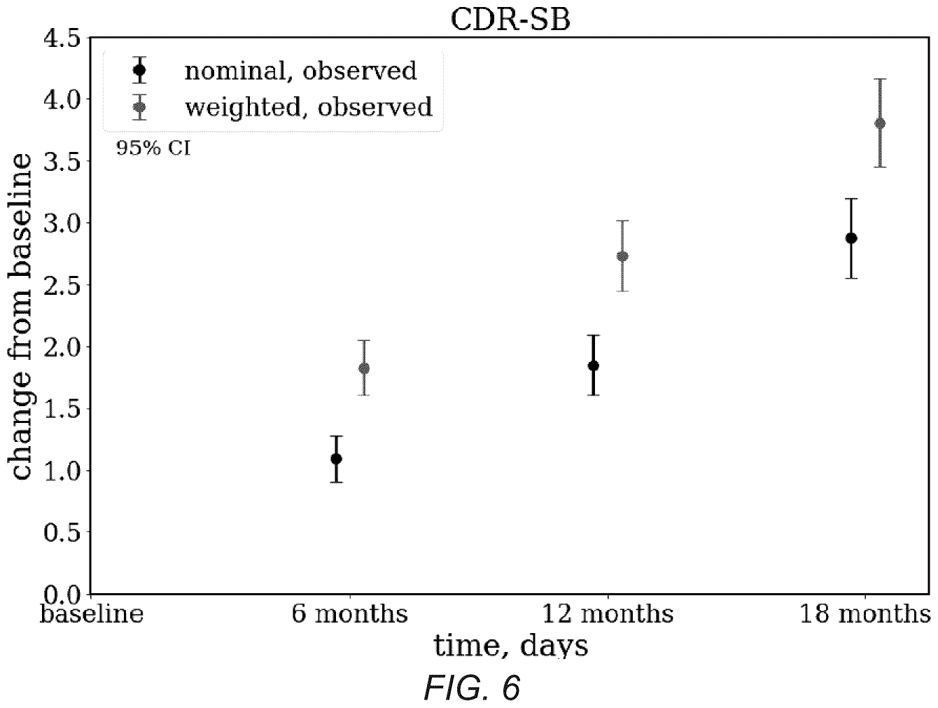

A graphical representation of the impact of weighted composite scores with respect to determining treatment effects and statistical power is illustrated in FIG. 6. The example from Alzheimer's Disease for the Clinical Dementia Rating assessment, where the nominal total score corresponds to a series of CDR Sum of Boxes (CDR-SB) measurements. The example depicts personalized composite scores (labeled “weighted, observed”) compared to nominal total scores. The personalized composite score is computed by optimizing the weights for each individual and applying the weights to their respective items. In this example, the weights are optimized (to the efficient frontier) further to the MSDR point, which minimizes the ratio of the resulting target mean to the standard deviation.

The impact of processes performed in accordance with numerous embodiments of the invention can, in many cases, be substantial. For example, FIG. 6 depicts a substantial increase in the impact of a given treatment (relative to baseline scores) when the composite scores have been optimized. This applies to multiple potential endpoints of the clinical trial, with a difference of approx. +0.6 points being detected for the optimized composite score after six months; and a difference of +0.9 points being detected for the optimized composite score after twelve months and eighteen months (relative to their respective nominal composite score equivalents). In this case, the weighted total scores show stronger progression than the nominal scores, reflecting the conclusion that weighted scores are more sensitive to treatment effects (i.e., possess higher signals relative to noise). The sensitivity to treatment effects, and hence the application to situations like clinical trials to increase power or make faster futility decisions, is an important consideration of this approach. Treatment effects that have constant shifts across items will behave differently than treatment effects where the effect is proportional to the item value.

While specific processes for optimizing composite scores are described above, any of a variety of processes can be utilized to update their respective weights as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

### III. SYSTEMS FOR DETERMINING TREATMENT EFFECTS

### A. Treatment Analysis System

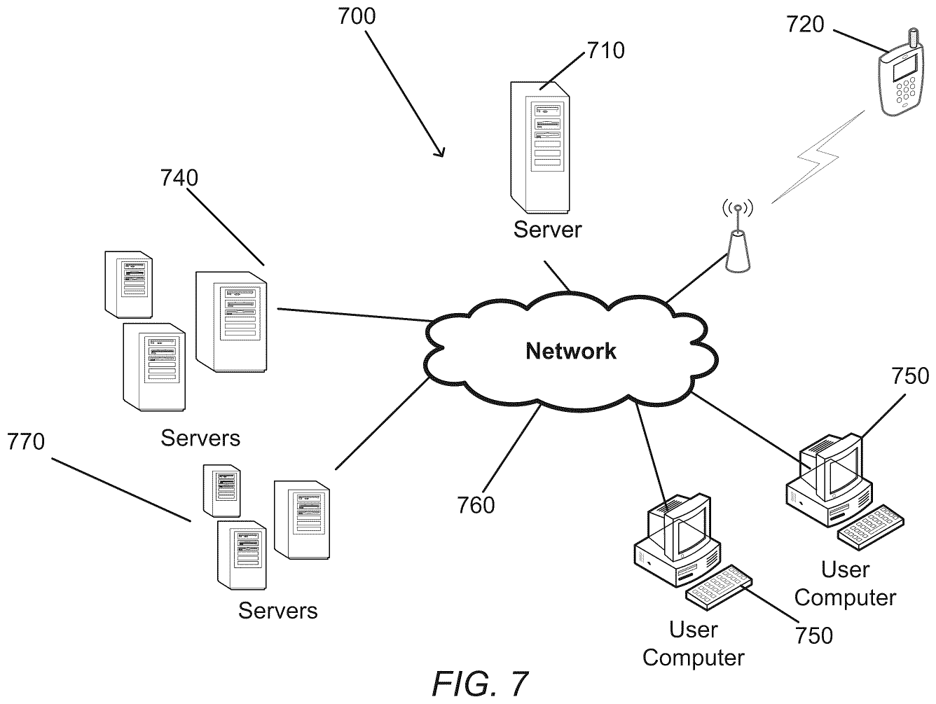

An example of a treatment analysis system that determines treatment effects in accordance with some embodiments of the invention is illustrated in FIG. 7. Network 700 includes a communications network 760. The communications network 760 is a network such as the Internet that allows devices connected to the network 760 to communicate with other connected devices. Server systems 710, 740, and 770 are connected to the network 760. Each of the server systems 710, 740, and 770 is a group of one or more servers communicatively connected to one another via internal networks that execute processes that provide cloud services to users over the network 760. One skilled in the art will recognize that a treatment analysis system may exclude certain components and/or include other components that are omitted for brevity without departing from this invention.

For purposes of this discussion, cloud services are one or more applications that are executed by one or more server systems to provide data and/or executable applications to devices over a network. The server systems 710, 740, and 770 are shown each having three servers in the internal network. However, the server systems 710, 740, and 770 may include any number of servers and any additional number of server systems may be connected to the network 760 to provide cloud services. In accordance with various embodiments of this invention, treatment analysis systems in accordance with various embodiments of the invention may be provided by a process being executed on a single server system and/or a group of server systems communicating over network 760.

Users may use personal devices 780 and 720 that connect to the network 760 to perform processes that determine treatment effects in accordance with various embodiments of the invention. In the shown embodiment, the personal devices 780 are shown as desktop computers that are connected via a conventional “wired” connection to the network 760. However, the personal device 780 may be a desktop computer, a laptop computer, a smart television, an entertainment gaming console, and/or any other device that connects to the network 760 via a “wired” connection. The mobile device 720 connects to network 760 using a wireless connection. A wireless connection is a connection that uses Radio Frequency (RF) signals, Infrared signals, and/or any other form of wireless signaling to connect to the network 760. In FIG. 7, the mobile device 720 is a mobile telephone. However, mobile devices 720 may be mobile phones, Personal Digital Assistants (PDAs), tablets, smartphones, and/or any other type of device that connects to network 760 via wireless connection without departing from this invention.

As can readily be appreciated, the specific computing system used to determine treatment effects is largely dependent upon the requirements of a given application and should not be considered as limited to any specific computing system(s) implementation.

### B. Treatment Analysis Element

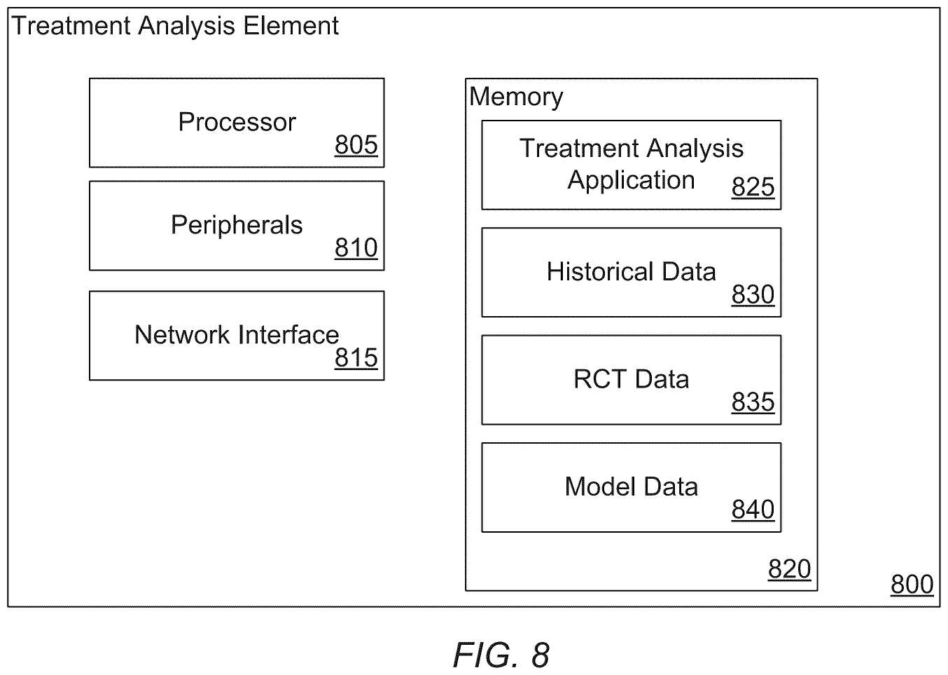

An example of a treatment analysis element that executes instructions to perform processes that determine treatment effects in accordance with various embodiments of the invention is illustrated in FIG. 8. Treatment analysis elements in accordance with many embodiments of the invention can include (but are not limited to) one or more of mobile devices, cloud services, and/or computers. Treatment analysis element 800 includes processor 805, peripherals 810, network interface 815, and memory 820. One skilled in the art will recognize that a treatment analysis element may exclude certain components and/or include other components that are omitted for brevity without departing from this invention.

The processor(s) 805 can include (but is not limited to) a processor, microprocessors, controller, and/or a combination of processors, microprocessors, and/or controllers that perform instructions stored in the memory 820 to manipulate data stored in the memory. Processor instructions can configure processors 805 to perform processes in accordance with certain embodiments of the invention.

Peripherals 810 can include any of a variety of components for capturing data, such as (but not limited to) cameras, displays, and/or sensors. In a variety of embodiments, peripherals can be used to gather inputs and/or provide outputs. Treatment analysis element 800 can utilize network interface 815 to transmit and receive data over a network based upon the instructions performed by processor 805. Peripherals 810 and/or network interfaces 815 in accordance with many embodiments of the invention can be used to gather data that can be used to determine treatment effects.

Memory 820 includes a treatment analysis application 825, historical data 830, RCT data 835, and model data 840. Treatment analysis applications in accordance with several embodiments of the invention can be used to determine treatment effects of an RCT, to design an RCT, and/or determine decision rules for treatments.

Historical data 830 in accordance with many embodiments of the invention can be used to pre-train generative models to generate potential outcomes for digital subjects and/or digital twins. In numerous embodiments, historical data 830 can include (but is not limited to) control arms from historical control arms, patient registries, electronic health records, and/or real-world data. In many embodiments, predictions from generative models can be compared to historical data 830 that was not used to train the models in order to obtain prior distributions capturing how well the predictions generalize to new populations.

In some embodiments, RCT data 835 can include panel data collected from subjects of RCTs. RCT data 835 in accordance with a variety of embodiments of the invention can be divided into control and treatment arms based on whether subjects received a treatment. In many embodiments, RCT data 835 can be supplemented with generated subject data. Generated subject data in accordance with a number of embodiments of the invention can include (but is not limited to) digital subject data and/or digital twin data.

In several embodiments, model data 840 can store various parameters and/or weights for generative models. Model data 840 in accordance with many embodiments of the invention can include data for models trained on historical data 830 and/or trained on RCT data 835. In several embodiments, pre-trained models can be updated based on RCT data 835 to generate digital subjects.

Although a specific example of a treatment analysis element 1000 is illustrated in this figure, any of a variety of treatment analysis elements can be utilized to perform processes for determining treatment effects similar to those described herein as appropriate to the requirements of specific applications in accordance with embodiments of the invention.

### C. Treatment Analysis Application

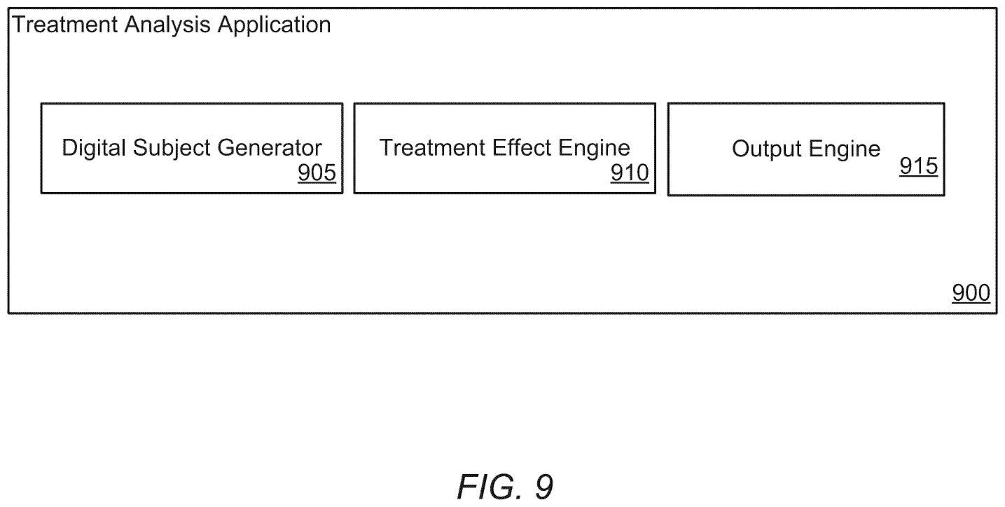

An example of a treatment analysis application for determining treatment effects in accordance with some embodiments of the invention is illustrated in FIG. 9. Treatment analysis applications 900 include, but are not limited to a digital subject generator 905, treatment effect engine 910, and output engine 915. One skilled in the art will recognize that a treatment analysis application 900 may exclude certain components and/or include other components that are omitted for brevity without departing from this invention.

Digital subject generators 905 in accordance with various embodiments of the invention can include generative models that can generate digital subject and/or digital twin data. Generative models in accordance with certain embodiments of the invention can be trained to generate potential outcome data based on characteristics of an individual and/or a population. Digital subject data in accordance with several embodiments of the invention can include (but is not limited to) panel data, outcome data, etc. In several embodiments, generative models can include (but are not limited to) traditional statistical models, generative adversarial networks, recurrent neural networks, Gaussian processes, autoencoders, autoregressive models, variational autoencoders, and/or other types of probabilistic generative models.

In various embodiments, treatment effect engines 910 can be used to determine treatment effects based on generated digital subject data and/or data from a RCT. In some embodiments, treatment effect engines 910 can use digital subject data from digital subject generators 905 to determine treatment effects in a variety of different applications, such as, but not limited to, comparing separate generative models based on data from the control and treatment arms of RCTs, supplementing control arms in RCTs, comparing predicted potential control outcomes with actual treatment outcomes, etc. Treatment effect engines 910 in accordance with many embodiments of the invention can be used to determine individualized responses to treatment. In certain embodiments, treatment effect engines 910 can determine biases of generative models of the digital subject generator and incorporate the biases (or corrections for the biases) in the treatment effect analyses.

Output engines 915 in accordance with several embodiments of the invention can provide a variety of outputs to a user, including (but not limited to) decision rules, treatment effects, generative model biases, recommended RCT designs, etc. In numerous embodiments, output engines 915 can provide feedback when the results of generative models of a digital subject generator 905 diverge from the RCT population. For example, output engines in accordance with certain embodiments of the invention can provide a notification when a difference between generated control outcomes for digital twins of subjects from a control arm and their actual control outcomes exceeds a threshold.

Although a specific example of a treatment analysis application is illustrated in this figure, any of a variety of Treatment analysis applications can be utilized to perform processes for determining treatment effects similar to those described herein as appropriate to the requirements of specific applications in accordance with embodiments of the invention.

Systems and techniques for evaluating treatment effects over time, are not limited to use for randomized controlled trials. Accordingly, it should be appreciated that applications described herein can be implemented outside the context of generative model architectures and in contexts unrelated to RCTs. Moreover, any of the systems and methods described herein with reference to FIGS. 1-9 can be utilized within any of the generative models described above.

Although specific methods of determining treatment effects are discussed above, many different methods of treatment analysis can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for optimizing a clinical trial configuration, the method comprising:
deriving a plurality of item scores for each of a plurality of subjects, wherein the plurality of item scores for a given subject of the plurality of subjects:
is based, at least in part, on a first set of subject data corresponding to a randomized control trial; and
answers a set of items from at least one medical evaluation;
identifying, from the plurality of item scores, at least one parameter, wherein the at least one parameter comprises an expected value for each of the set of items across the plurality of subjects;
optimizing, from the at least one parameter, at least one vector of item weights, wherein:
the at least one vector of item weights is derived using a mean-variance analysis; and
each item weight of the at least one vector of item weights comprises a non-negative number;
determining, for each of the plurality of subjects, an initial composite score, wherein:
the initial composite score is determined from:
one of the at least one vector of item weights; and
the plurality of item scores;
the mean-variance analysis optimizes the at least one vector of item weights by deriving the item weights that minimize a variance for a resulting collection of composite scores; and
the resulting collection of composite scores comprises the initial composite score determined for each of the plurality of subjects; and
applying the resulting collection of composite scores as a second set of subject data used in implementing a clinical trial, wherein applying the resulting collection of composite scores comprises:
determining, based on the resulting collection of composite scores, at least one decision rule for the clinical trial; and
deriving, based on the at least one decision rule, one or more of:
a desired type-I error rate for the clinical trial; or
a desired type-II error rate for the clinical trial.

2. The method of claim 1, wherein determining, for each of the plurality of subjects, the initial composite score is performed non-linearly.

3. The method of claim 1, wherein the at least one parameter further comprises a set of one or more covariance measurements corresponding to the at least one vector of item weights.

4. The method of claim 3, wherein the set of one or more covariance measurements comprises a covariance matrix corresponding to the expected value for each of the set of items across the plurality of subjects.

5. The method of claim 1, wherein each vector of the at least one vector of item weights is uniquely generated for the given subject of the plurality of subjects.

6. The method of claim 5, wherein:
the mean-variance analysis is performed at least in part based on a pre-determined target mean composite score; and
summing every item weight of the at least one vector of item weights produces a singular numerical constant.

7. The method of claim 6, wherein the mean-variance analysis comprises performing one or more of:
minimizing a variance across initial composite scores predicted for the plurality of subjects; or
maximizing a ratio of a target mean to a standard deviation across initial composite scores predicted for the plurality of subjects.

8. The method of claim 1, wherein:
the plurality of item scores is derived based on digital subject data generated by a set of one or more generative models; and
at least one of the set of one or more generative models is a neural network trained, at least in part, based on a set of historical data, comprising one or more of: control arm data from historical control arms, patient registries, electronic health records, or real world data.

9. The method of claim 8, wherein the clinical trial is based, at least in part, on outcome data generated from the set of one or more generative models.

10. The method of claim 1, wherein implementing the clinical trial comprises:
using the second set of subject data as baseline data for the clinical trial;
during at least one future time point in the clinical trial, obtaining subsequent data for the clinical trial, wherein:
obtaining the subsequent data for the clinical trial comprises determining, for each of the plurality of subjects, an additional composite score; and
the additional composite score is determined from:
the same one, of the at least one vector of item weights, used to determine the initial composite score; and
a plurality of subsequent item scores; and
adding the additional composite score determined for each of the plurality of subjects to the resulting collection of composite scores.

11. A non-transitory machine-readable medium comprising instructions that, when executed, are configured to cause a processor to perform a process for optimizing a clinical trial configuration, the process comprising:
deriving a plurality of item scores for each of a plurality of subjects, wherein the plurality of item scores for a given subject of the plurality of subjects:
is based, at least in part, on subject data corresponding to a randomized control trial; and
answers a set of items from at least one medical evaluation;
identifying, from the plurality of item scores, at least one parameter, wherein the at least one parameter comprises an expected value for each of the set of items across the plurality of subjects;
optimizing, from the at least one parameter, at least one vector of item weights, wherein:
the at least one vector of item weights is derived using a mean-variance analysis; and
each item weight of the at least one vector of item weights comprises a non-negative number;
determining, for each of the plurality of subjects, an initial composite score, wherein:
the initial composite score is determined from the at least one vector of item weights and the plurality of item scores;
the mean-variance analysis optimizes the at least one vector of item weights by deriving the item weights that minimize a variance for a resulting collection of composite scores; and
the resulting collection of composite scores comprises the initial composite score determined for each of the plurality of subjects; and
applying the resulting collection of composite scores as a second set of subject data used in implementing a clinical trial, wherein applying the resulting collection of composite scores comprises:
determining, based on the resulting collection of composite scores, at least one decision rule for the clinical trial; and
deriving, based on the at least one decision rule, one or more of:
a desired type-I error rate for the clinical trial; or
a desired type-II error rate for the clinical trial.

12. The non-transitory machine-readable medium of claim 11, wherein determining, for each of the plurality of subjects, the initial composite score is performed non-linearly.

13. The non-transitory machine-readable medium of claim 11, wherein the at least one parameter further comprises a set of one or more covariance measurements corresponding to the at least one vector of item weights.

14. The non-transitory machine-readable medium of claim 13, wherein the set of one or more covariance measurements comprises a covariance matrix corresponding to the expected value for each of the set of items across the plurality of subjects.

15. The non-transitory machine-readable medium of claim 11, wherein each vector of the at least one vector of item weights is uniquely generated for the given subject of the plurality of subjects.

16. The non-transitory machine-readable medium of claim 15, wherein:
the mean-variance analysis is performed at least in part based on a pre-determined target mean composite score; and
summing every item weight of the at least one vector of item weights produces a singular numerical constant.

17. The non-transitory machine-readable medium of claim 16, wherein the mean-variance analysis comprises performing one or more of:
minimizing a variance across initial composite scores predicted for the plurality of subjects; or
maximizing a ratio of a target mean to a standard deviation across initial composite scores predicted for the plurality of subjects.

18. The non-transitory machine-readable medium of claim 11, wherein:
the plurality of item scores is derived based on digital subject data generated by a set of one or more generative models; and
at least one of the set of one or more generative models is a neural network trained, at least in part, based on a set of historical data, comprising one or more of: control arm data from historical control arms, patient registries, electronic health records, or real world data.

19. The non-transitory machine-readable medium of claim 18, wherein the clinical trial is based, at least in part, on outcome data generated from the set of one or more generative models.

20. The non-transitory machine-readable medium of claim 11, wherein implementing the clinical trial comprises:
using the second set of subject data as baseline data for the clinical trial;
during at least one future time point in the clinical trial, obtaining subsequent data for the clinical trial, wherein:
obtaining the subsequent data for the clinical trial comprises determining, for each of the plurality of subjects, an additional composite score; and
the additional composite score is determined from:
the same one, of the at least one vector of item weights, used to determine the initial composite score; and
a plurality of subsequent item scores; and
adding the additional composite score determined for each of the plurality of subjects to the resulting collection of composite scores.
